(19) United States
(12) Patent Application Publication
(10) Pub. No.: US 2012/0039954 A1
(43) Pub. Date: Feb. 16, 2012

(54) METHOD OF TREATING INSOMNIA

(75) Inventors: Gary Cupit, Basking Ridge, NJ (US); Anne McCormick, Madison, NJ (US); Mary Osbakken, Philadelphia, PA (US); Christine Blumhardt, Strafford, PA (US)

(73) Assignee: SOMNUS THERAPEUTICS, INC., Bedminster, NJ (US)

(21) Appl. No.: 13/139,881

(22) PCT Filed: Dec. 15, 2009
(86) PCT No.: PCT/US09/68053

§ 371 Date: Nov. 2, 2011

Related U.S. Application Data
(60) Provisional application No. 61/201,904, filed on Dec. 15, 2008.
(60) Provisional application No. 61/201,917, filed on Dec. 15, 2008.
(60) Provisional application No. 61/201,853, filed on Dec. 15, 2008.
(60) Provisional application No. 61/201,844, filed on Dec. 15, 2008.

(51) Int. Cl. A61K 31/519; A61P 25/20; A61K 9/00

(52) U.S. Cl. 424/400; 514/259.3

(57) ABSTRACT

A method of treating insomnia comprising administering to a subject a formulation including zaleplon, wherein the formulation is adapted to release the zaleplon after a lag time of at least about one hour after administration of the formulation, and during which substantially no drug substance is released; provide a time of peak plasma concentration of about 3 hours to about 6 hours after administration; provide an elimination half-life after the time of peak plasma concentration of about 0.5 hours to about 0.3 hours; and provide an area under the curve of about 70 ng·h/mL to about 90 ng·h/mL.

### BACKGROUND

The present invention is concerned with methods and compositions for treating insomnia in human subjects.

Many pathologies or conditions are related to abnormalities within diurnal rhythms. Insomnia is such a condition. However, whereas insomnia is a very prevalent condition it is generally considered among physicians that many people are amenable to pharmacologic intervention to help ameliorate their problems. When assessing the symptoms of insomnia, physicians have found that they fall generally within the categories of i) latency to sleep, ii) duration of sleep, iii) disturbed patterns of sleep, i.e. frequent nocturnal wakening events, and iv) residual hangover effects upon awakening such as drowsiness and impairment of cognitive and motor functions.

Early treatments for insomnia commonly employed central nervous system (CNS) depressants such as barbiturates. These compounds typically have long half lives and have a well-known spectrum of side effects, including lethargy, confusion, depression and next day hangover effects. In addition, chronic use has been associated with a high potential for addiction involving both physical and psychological dependence.

Treatments moved away from barbiturates and other CNS depressants toward the benzodiazepine class of sedative-hypnotic agents. This class of compounds produces a calming effect that results in a sleep-like state in humans and animals, with a greater safety margin than prior hypnotics. However, many benzodiazepines possess side effects that limit their usefulness in certain patient populations. These problems include synergy with other CNS depressants (especially alcohol), the development of tolerance upon repeat dosing, rebound insomnia following discontinuation of dosing, hangover effects the next day and impairment of psychomotor performance and memory.

More recent treatments for insomnia have used non-benzodiazepine compounds. Ambien (zolpidem), Sonata (zaleplon) are examples of approved drug products. Zaleplon, also known as N-[3-(3-cyanopyrazole[1,5-a]pyrimidin-7-yl)phenyl]-N-ethylacetamide, is a pyrazolopyrimidine hypnotic that binds selectively to the benzodiazepine type I site on the GABA-A (γ-aminobutyric acid, type A) receptor complex. Other non-benzodiazepine compounds useful in the treatment of insomnia are known in the literature and can be employed in the present invention. What is clear, however, is that there is still hesitance on the part of patients and physicians with regard to the use of sedatives and other CNS active agents in a chronic setting. Despite huge improvements in available drug substances, pharmacological intervention cannot rely solely on the properties inherent to these drug substances alone. The way in which such drug substances are formulated may largely influence their efficacy, side-effect profiles, and ultimately the acceptance by both patients and physicians alike.

### SUMMARY OF THE INVENTION

According to some embodiments, a method of treating insomnia includes administering to a subject a formulation comprising zaleplon, wherein the formulation is adapted to: (1) release the zaleplon after a lag time of at least about one hour after administration of the formulation, and during which substantially no drug substance is released; (2) provide a time of peak plasma concentration of about 3 hours to about 6 hours after administration; (3) provide an elimination half-life after the time of peak plasma concentration of about 0.5 hours to about 0.3 hours; and (4) provide an area under the curve of about 70 ng·h/mL to about 90 ng·h/mL.

In some embodiments, the lag time is at least about 1.5 hours. In some embodiments, less than about 10% of the zaleplon is released during the lag time. In certain embodiments, the formulation provides maximum sedation about 3 hours to about 5 hours after administration of the formulation. In some embodiments, the formulation provides no residual side effects about 8 hours post-dosing.

In some embodiments, the time of peak plasma concentration is about 3.75 hours to about 5.25 hours after administration; or about 4 hours to about 5 hours after administration. In some embodiments, the elimination half-life is about 0.5 hours to about 2.5 hours; or about 1 hour to about 2 hours. In some embodiments, the area under the curve is about 75 ng·h/mL to about 85 ng·h/mL; or about 78 ng·h/mL to about 85 ng·h/mL.

In some embodiments, the formulation includes a core and a shell. In certain embodiments, the core includes zaleplon, hydroxypropylmethyl cellulose, and lactose monohydrate. In some embodiments, the core includes about 20% to about 30% zaleplon; or about 25% zaleplon. In some embodiments, the core includes about 25% to about 35% hydroxypropylmethyl cellulose; or about 31.4% hydroxypropylmethyl cellulose. In some embodiments, the core includes about 25% to about 35% lactose monohydrate; or about 31.4% lactose monohydrate. In some embodiments, the core includes about 1% to about 15% polyvinylpyrrolidone; or about 5% polyvinylpyrrolidone.

In some embodiments, the shell includes about 35% to about 45% dibasic calcium phosphate; or about 38.9% dibasic calcium phosphate. In some embodiments, the shell includes glyceryl behenate in an amount of about 15% to about 25%; or about 21.1%. In some embodiments, the shell includes about 1% to about 15% polyvinylpyrrolidone; or about 6.53% polyvinylpyrrolidone. In some embodiments, the shell includes about 1% to about 15% microcrystalline cellulose; or about 10% microcrystalline cellulose.

In certain embodiments, the formulation includes about 5 mg to about 50 mg zaleplon; or about 15 mg zaleplon.

According to some embodiments, the formulation includes a core and a shell, wherein the core includes about 20% to about 30% zaleplon; about 25% to about 35% hydroxypropylmethyl cellulose; about 25% to about 35% lactose monohydrate; about 1% to about 15% polyvinylpyrrolidone; and wherein the shell includes about 35% to about 45% dibasic calcium phosphate; about 15% to about 25% glyceryl behenate; about 1% to about 15% polyvinylpyrrolidone; and about 1% to about 15% microcrystalline cellulose.

### DESCRIPTION OF THE DRAWINGS

The accompanying drawings, which are included to provide further understanding of the disclosure and are incorporated in and constitute a part of this specification, illustrate embodiments of the disclosure and, together with the description, serve to explain the principles of the disclosure.

In the drawings:

FIG. 1 shows a 2-dimensional representation of a dosage form of some embodiments of the present invention;

FIG. 2 shows the release profiles of several tablets of some embodiments of the present invention; and

FIG. 3 illustrates the release of Zaleplon from the formulations of some embodiments of the present invention.

### DETAILED DESCRIPTION

According to some embodiments of the present invention, a method of treating insomnia includes administering to a subject a formulation including a drug substance, wherein the formulation is adapted to release the drug substance after a lag time. The formulation may provide consistent active drug concentrations thereafter, with rapid decline after the time of peak plasma concentration.

Release Profile/Lag Time

Certain sedatives are commonly available or are in development in the form of immediate release dosage forms. As is well known in the art, immediate release dosage forms provide a burst of drug substance shortly after ingestion to induce rapid onset of sleep. Whereas such dosage forms address the latency to sleep problem, unless the drug substance has a long half life, in order to maintain effective blood plasma concentration levels over an extended period of time, patients experiencing short sleep duration or frequent nocturnal awakening events will need to take further dosage forms during the night to maintain sleep.

Modified release dosage forms produce an initial burst of drug substance to induce rapid onset of sleep, and continue to release drug substance in a controlled manner to maintain effective plasma concentrations over an extended period of time to improve sleep maintenance. A potential disadvantage of this approach is the time to clearance of the active substance from a patient's system. Drug substance still present at effective levels can cause hangover effects upon wakening.

A particular modified release dosage form is described in U.S. Pat. No. 6,485,746. In this patent there is described a formulation of a sedative-hypnotic compound that provides a pulsatile release profile in vivo whereby upon administration the drug substance is released rapidly to provide a maximum plasma concentration within 0.1 to 2 hours following administration. Thereafter, plasma concentration passes through a minimum at about 2 to 4 hours post administration, before a second pulse delivers a second maximum plasma concentration at about 3 to 5 hours. Finally, after 8 hours there remains a plasma concentration that represents no more than 20% of the plasma concentration of the second maximum.

Existing formulations and those in development are only concerned with improving the quality of sleep and the prevention of hangover effects. However, such formulations fail to address the problems that sedatives can create to a patient's presleep routine. The rapid onset of drowsiness, and the concomitant disruption of presleep activities such as reading and watching television, may result in increased hesitance of physicians to prescribe a drug, and poorer patient compliance.

Sedation affecting pre-sleep routines is an unpleasant aspect of insomnia medications, made more so when one considers that a high proportion of insomnia sufferers do not complain of problems falling asleep, but are only afflicted by short sleep duration and frequent nocturnal awakening events. Furthermore, there is evidence suggesting a significant placebo effect associated with therapies intended to initiate a rapid onset of sleep.

Despite the increased activity in the development of therapeutics in this area, there remains a need to offer patients a dosage form that can be taken before bedtime that not only provides extended sleep duration and reduces or eliminates nocturnal awakening events, but which leaves patients free to go about pre-sleep activities unsedated.

In some embodiments, the present invention provides in a first aspect a method of treating insomnia in a patient in need thereof, comprising administering a dosage form containing a drug substance useful in treating insomnia, the dosage form being adapted to release said drug substance after a lag time during which no, or substantially no, drug substance is released, the lag time being about at least one hour after administration of the dosage form.

In some embodiments, the dosage form used in the method of the present invention is adapted to release the active drug substance in a time-dependent manner, i.e., after a pre-determined lag time. In certain embodiments, no extrinsic changes in the environment, such as a change in pH or temperature, are required in order to prompt release of the drug substance from the dosage form after the pre-determined lag time. In some embodiments, the lag time may be from about 1 hour to about 4 hours, about 1 hour to about 2 hours, or about 2 hours to about 3 hours.

The pH of the gastric tract can differ markedly depending on whether a patient is in a fed or fasted state. Accordingly, to achieve a reliable pre-determined lag time, the release of said drug substance from the dosage form may be pH-independent. In some embodiments, during the pendency of lag time any drug substance that is released is in such small amounts that effective blood plasma levels of the drug substance are not reached. In certain embodiments, drug substance release is less than about 10% by weight, less than about 5%, less than about 2%, or less than about 1%.

In some embodiments, following the expiry of the lag time, the drug substance is released from the dosage form. The drug substance may for example be released rapidly (immediate release) or may be released slowly over a period of time (modified release). In some embodiments, the drug substance may be released in a non-pulsatile manner. Thus, the drug substance may be released from the dosage form at a steady or continuous rate. Lag time can be measured in vitro using dissolution methods and apparatus generally known in the art. The United States Pharmacopoeia describes several such methods.

In some embodiments of the present invention, there is provided a method of treating insomnia in a patient in need thereof comprising administering a dosage form containing a drug substance useful in treating insomnia, the dosage form being adapted to release said drug substance after a lag time during which no, or substantially no, drug substance is released, the lag time being about at least one hour after administration of the dosage form, which dosage form is adapted to obtain a controlled release of said drug substance in vitro when measured by the USP Paddle Method (type II apparatus) at 100 rpm, in 1000 ml of an aqueous medium such that during said lag time, not more than about 10% of drug substance is released.

In some embodiments of the present invention, there is provided a method of treating insomnia in a patient in need thereof comprising administering a dosage form containing a drug substance useful in treating insomnia, the dosage form being adapted to release said drug substance after a lag time during which no, or substantially no, drug substance is released, the lag time being about at least one hour after administration of the dosage form, which dosage form is adapted to obtain a controlled release of said drug substance in vitro when measured by the USP Paddle Method (type II apparatus) at 100 rpm at 37° C. in 1000 ml of (a) 0.1M HCl and phosphate buffer (pH 6.8) or (b) 0.02% sodium lauryl sulphate in 500 ml distilled water or (c) purified water, such that during said lag time not more than 10% of drug substance is released.

In certain embodiments, in a method according to the present invention a dosage form is adapted to obtain a controlled release of said drug substance in vitro when measured by the USP Paddle Method (type II apparatus) at 100 rpm, in 1000 ml of an aqueous medium such that during said lag time not more than about 10% of drug substance is released, at least about 25% to about 60% is released within 5 hours, and at least about 80% is released after 7 hours.

In some embodiments, in a method according to the present invention a dosage form is adapted to obtain a controlled release of said drug substance in vitro when measured by the USP Paddle Method (type II apparatus) at 100 rpm at 37° C. in 1000 ml of (a) 0.1M HCl and phosphate buffer (pH 6.8) or (b) 0.02% sodium lauryl sulphate in 500 ml distilled water or (c) purified water, in an aqueous medium such that during said lag time not more than about 10% of drug substance is released, at least about 25% to about 60% is released within 5 hours, and at least about 80% is released after 7 hours.

Pharmacokinetic Profile

The activity of the inventive formulations may be dependent on their pharmacokinetic behavior. This pharmacokinetic behavior defines the drug concentrations and period of time over which a subject is exposed to the drug. In the case of insomnia treatment drugs, it may be advantageous for a formulation to be adapted to provide a lag time before release of the drug, a consistent drug concentration after release, and a rapid decline in drug concentration after the peak plasma concentration.

In general, several parameters may be used to describe drug pharmacokinetics. Time from administration to peak plasma concentration, elimination half-life, and area under the curve (AUC) are examples. The elimination half-life is the time required for half of the administered drug to be removed from the plasma. The AUC is a measure of plasma drug levels over time and provides an indication of the total drug exposure.

In some embodiments, in a method according to the present invention a dosage form is adapted to provide a time from administration to peak plasma concentration of about 3 hours to about 6 hours; about 3.25 hours to about 5.25 hours; about 3.5 hours to about 5 hours; about 3.75 hours to about 5 hours; about 3.75 hours to about 4.5 hours; about 3.75 hours to about 4.25 hours; about 4.5 hours to about 5.5 hours; about 4.75 hours to about 5.25 hours; or about 4 hours to about 5 hours. In some embodiments, in a method according to the present invention a dosage fowl is adapted to provide a time from administration to peak plasma concentration of about 3 hours; about 3.1 hours; about 3.2 hours; about 3.3 hours; about 3.4 hours, about 3.5 hours; about 3.6 hours, about 3.7 hours; about 3.8 hours; about 3.9 hours; about 4 hours; about 4.1 hours; about 4.2 hours; about 4.3 hours; about 4.4 hours; about 4.5 hours; about 4.6 hours; about 4.7 hours; about 4.8 hours; about 4.9 hours; about 5 hours; about 5.1 hours; about 5.2 hours; about 5.3 hours; about 5.4 hours; about 5.5 hours; about 5.6 hours; about 5.7 hours; about 5.8 hours; about 5.9 hours; or about 6 hours.

In some embodiments, in a method according to the present invention a dosage form is adapted to provide a rapid decline in plasma concentrations after the peak plasma concentration. In some embodiments, in a method according to the present invention a dosage form is adapted to provide a decline in plasma concentration after the peak plasma concentration with an elimination half life of about 0.5 hours to about 3 hours; about 0.5 hours to about 2.5 hours; or about 1 hour to about 2 hours. In some embodiments, in a method according to the present invention a dosage form is adapted to provide a decline in plasma concentration after the peak plasma concentration with an elimination half life of about 0.5 hours; about 0.6 hours; about 0.7 hours; about 0.75 hours; about 0.8 hours; about 0.9 hours; about 1 hour; about 1.1 hours; about 1.2 hours; about 1.25 hours; about 1.3 hours; about 1.4 hours; about 1.5 hours; about 1.6 hours; about 1.7 hours; about 1.75 hours; about 1.8 hours; about 1.9 hours; about 2 hours; about 2.1 hours; about 2.2 hours; about 2.25 hours; about 2.3 hours; about 2.4 hours; or about 2.5 hours.

In some embodiments, in a method according to the present invention a dosage form is adapted to provide increased plasma drug levels over time, represented by area under the curve (“AUC”). In some embodiments, in a method according to the present invention, a dosage form is adapted to provide an AUC of about 60 n·gh/mL to about 100 n·gh/mL; about 65 n·gh/mL to about 95 n·gh/mL; about 70 n·gh/mL to about 90 n·gh/mL; about 75 n·gh/mL to about 85 n·gh/mL; or about 78 n·gh/mL to about 85 n·gh/mL. In some embodiments, in a method according to the present invention, a dosage form is adapted to provide an AUC of about 60 n·gh/mL; about 60 n·gh/mL; about 60 n·gh/mL; about 61 n·gh/mL; about 62 n·gh/mL; about 63 n·gh/mL; about 64 n·gh/mL; about 65 n·gh/mL; about 66 n·gh/mL; about 67 n·gh/mL; about 68 n^{SM}gh/mL; about 69 n·gh/mL; about 70 n·gh/mL; about 71 n·gh/mL; about 72 n·gh/mL; about 73 n·gh/mL; about 74 n·gh/mL; about 75 n·gh/mL; about 76 n·gh/mL; about 77 n·gh/mL; about 78 n·gh/mL; about 79 n·gh/mL; about 80 n·gh/mL; about 81 n·gh/mL; about 82 n·gh/mL; about 83 n·gh/mL; about 84 n·gh/mL; about 85 n·gh/mL; about 86 n·gh/mL; about 87 n·gh/mL; about 88 n·gh/mL; about 89 n·gh/mL; about 90 n·gh/mL; about 91 n·gh/mL; about 92 n·gh/mL; about 93 n·gh/mL; about 94 n·gh/mL; about 95 n·gh/mL; about 96 n·gh/mL; about 97 n·gh/mL; about 98 n·gh/mL; about 99 n·gh/mL; about 100 n·gh/mL; about 83.2 n·gh/mL; about 83.1 n·gh/mL; or about 79.5 n·gh/mL.

Lag Time

The invention further provides a dosage form useful in the above methods. In some embodiments, the dosage form is provided as a unit (single-component) dose. From the perspective of products for the treatment of insomnia that work by delivering an immediate pulse of drug substance to combat latency to sleep problems, the method of administration involving a lag time is counter-intuitive, and may provide certain advantages over existing therapies. For example, a patient may be free to go about its pre-sleep activities without feeling sedated.

Although the dosage form in accordance with some embodiments of the present invention delivers the drug substance after a lag time, given the significant placebo effect referred to above it may be useful for treating or addressing sleep latency as well as wakening events.

Other advantages relate to the biological processes associated with the sleep. The so-called “homeostatic process” is believed to be a primary driving force in creating in patients the need for sleep. For an individual having a bed time of around 11 p.m., this drive weakens in the early morning hours, e.g., around 3 a.m., and is further exacerbated by a circadian alert pulse around 5 a.m. that is believed to be an additional driver to wakefulness for patients. A lag time before drug release can ensure that peak plasma concentrations are reached several hours into the sleep cycle when nocturnal awakening events are likely to occur. By coinciding drug release and therefore maximum plasma concentrations with these processes occurring in the early morning hours, it may be possible to use lower doses of drug substances than would otherwise be needed using conventional sustained release dosage forms that must contain a significant amount of drug sub stance to provide the initial drug burst to arrest sleep latency problems.

Still further, many drug substances are metabolized by cytochrome CYP450 isoform 3A4, and this enzyme is present in relatively high concentrations in higher regions of the gastro-intestinal (GI) tract. In some embodiments, a dosage form exhibiting a lag time may pass further down the GI tract before delivering drug substance in a region of lower CYP P450 activity, thereby potentially increasing the efficacy of the released drug substance. The Eront-line sedative hypnotic, zaleplon, is such a drug substance that is metabolized by CYP P450.

A dosage form in accordance with some embodiments of the present invention can deliver a drug substance such that a peak plasma concentration occurs around 3 a.m. in the morning (that is, around 4-5 hours after administration). Furthermore, in certain embodiments, using commonly available sustained release excipients (as will be further described herein below), drug substance plasma concentrations may be maintained at effective levels though 3 a.m. to coincide with the weakening homeostatic process and through 5 a.m. to coincide with a circadian alert pulse mentioned above.

In some embodiments, a formulation may be adapted to release a drug substance after a lag time of about 1 hour to about 4 hours. In some embodiments, a formulation may be adapted to release a drug substance after a lag time of about 0.5 hours; about 0.6 hours; about 0.7 hours; about 0.8 hours; about 0.9 hours; about 1 hour; about 1.1 hours; about 1.2 hours; about 1.3 hours; about 1.4 hours; about 1.5 hours; about 1.6 hours; about 1.7 hours; about 1.8 hours; about 1.9 hours; about 2 hours; about 2.1 hours; about 2.2 hours; about 2.3 hours; about 2.4 hours; about 2.5 hours; about 2.6 hours; about 2.7 hours; about 2.8 hours; about 2.9 hours; about 3 hours; about 3.1 hours; about 3.2 hours; about 3.3 hours; about 3.4 hours; about 3.5 hours; about 3.6 hours; about 3.7 hours; about 3.8 hours; about 3.9 hours; or about 4 hours.

Shortened Sleep Pattern

Certain dosage forms described in the art are intended to achieve an extended sleep period of 8 hours. However, it is not always advantageous to deliver such an extended sleep pattern. In some instances, individuals may desire only to sleep for a short number of hours, e.g. 5 to 6 hours, before having to waken refreshed and alert. For such patients, it may not be considered advantageous to suppress the circadian alert pulse.

The dosage forms useful in the method of the some embodiments of the present invention are able to release a drug substance after a lag time in order to provide effective plasma concentrations of drug substance in order to coincide with the weakening homeostatic drive, and then permit the plasma levels to decay in a controllable manner to ensure a plasma levels are below effective levels between about 6 to 8 hours after administration, thereby avoiding or reducing the so-called “hangover effect”.

In general, the ability to avoid hangover effects, even after a relatively short sleep duration, e.g. of the order of 5 to 6 hours, may be more easily achieved by employing sedatives with short half lives. In general, a short-acting sedative is a compound that has a detectable sedative effect in any standard assay, with a mean plasma half-life of the compound of less than about 2 hours. In example includes but is not limited to zaleplon, which has a half life of about 1 hour; eszopiclone, zolpidem, indiplon, gaboxedol and ramelteon.

In some embodiments, the use of a short acting sedative in combination with the targeted dosing afforded by the dosage forms described herein, provides patients with the possibility of having relatively short sleep intervals and still wake up without experiencing hangover effects, or reduced hangover effects.

Composition

Drug Substances

Drug substances for use in some embodiments of the present invention may be any of those substances known to be useful for treating insomnia. Examples of useful classes of drug substances may include but are not limited to benzodiazepine receptor agonists; antihistamines; GABA A receptor agonists; imidazopyridines; Ureides; tertiary acetylinic alcohols; pipendine derivatives; GABA receptor agonists; and melatonin 1 receptor agonists.

Particular drug substances that may be useful in some embodiments of the present invention include but are not limited to Brotizolam, Lormetazepam, Loprazolam, Flunitrazepam, Nitrazepam, Estazolam, Flurazepam, Loprazolarn, Lonnetazepam, Midazolam, Nitrazepam, Nordazepam, Quazepam, Temazepam, Triazolam, Doxylamine, Diphenhydramine, Promethazine, Niaprazine, Clomethiazole, Paraldehyde, Chloral Hydrate, Triclofos, Zaleplon, Zolpldern, Acetylcarbromal, Ethchlorvynol, Niaprazine, Tiagabine, Glutethimide, Zopiclone, Eszopiclone, Ramelteon, Agomelatine, Indiplon, Eplivanserin, Lirequinil and Gaboxadol. Other substances known in the art by their internal code names may include Anph 101, Th 9507, Ly 156735, Org 4420, Ngd 963 and EMR 622 18. In some embodiments, a formulation includes zaleplon.

The amount of drug substance that may be employed will depend upon the type of drug substance, the type and severity of the condition to be treated, and the patient's medical history, age and weight. However, generally speaking drug substances may be administered in amounts to achieve a dose of from about 5 mg to about 50 mg per day, or about 10 mg to about 50 mg per day.

A unit dosage form for use in the method according to certain embodiments of the present invention may contain about 5 mg to about 50 mg of zaleplon; about 5 mg to about 25 mg of zaleplon; or about 10 mg to about 20 mg zaleplon. A unit dosage form for use in the method according to certain embodiments of the present invention may contain zaleplotn in an amount of about 5 mg; about 6 mg; about 7 mg; about 8 mg; about 9 mg; about 10 mg; about 11 mg; about 12 mg; about 13 mg; about 14 mg; about 15 mg; about 16 mg; about 17 mg; about 18 mg; about 19 mg; about 20 mg; about 21 mg; about 22 mg; about 23 mg; about 24 mg; about 25 mg; about 30 mg; about 35 mg; about 40 mg; about 45 mg; or about 50 mg.

Dosage forms for the administration of a drug substance to improve sleep patterns in patients suffering with insomnia may take a variety of forms that are capable of presenting the drug substance in bioavailable form in effective amounts.

Release Controlling Agent

In some embodiments, a dosage form contains one or more drug substances and a release controlling agent.

In some embodiments, the release controlling agent may be in a matrix in which the drug substance is dissolved or dispersed. Alternatively, the release controlling agent may be in a layer or coating surrounding a drug substance-containing matrix. When the release controlling agent is in the layer or coating, the matrix may also contain a release controlling agent, or it may be adapted for immediate release of the drug substance.

In some embodiments, the selection of appropriate matrix and/or coating materials aids in accurately controlling the lag time, as well as ensuring that all, or substantially all, of the drug substance upon expiry of the lag time is released at a desired rate to achieve extended sleep patterns and eliminate or reduce nocturnal awakening events.

In some embodiments, a coating material includes little or no swellable or gellable materials. Examples of such materials include but are not limited to cellulose ethers or cellulosic derivatives such as hydroxyalkyl celluloses, e.g. hydroxypropylmethyl cellulose, or carboxyalkylcelluloses and the like. Such materials may form gels which exert a release-controlling effect by forming an erodible barrier through which drug substances may diffuse. Such materials may result unreliable lag times and in some embodiments are avoided in amounts that exert a release-controlling effect. The release-controlling properties of such materials may be evident when they are employed in amounts of about 10% or greater. In some embodiments, if any of the aforementioned materials are employed as coating materials they may be used in small amounts, e.g. less than about 10%, less than about 5%, or less than about 1%.

In certain embodiments, a release controlling agent may include water-insoluble or poorly water soluble hydrophobic materials, such as waxy and insoluble excipients. In some embodiments, such excipients act by permitting ingress of aqueous physiological media through faults and channels in the bulk materials. Release controlling agents may include but are not limited to hydrophilic and/or hydrophobic materials, such as gums, natural and synthetic waxes such as beeswax, glycowax, castor wax and carnauba wax, shellac, and mineral and vegetable oils such as hydrogenated castor oil, hydrogenated vegetable oil, polyalkylene glycols, long chain (e.g. about 8 to about 50 carbon atoms) substituted or unsubstituted hydrocarbon such as fatty acids and fatty alcohols, or glyceryl esters of fatty acids.

Release controlling agents may be present in the dosage form in amounts depending on the desired release profile. In some embodiments, such agents may be present in amounts of about 1% to about 99% by weight of the dosage form.

Excipients

In addition to the above ingredients, in some embodiments a dosage form may also contain other excipients commonly employed in oral dosage foi ins such as diluents, lubricants, binders such as alkyl celluloses such as ethyl cellulose, granulating aids, colorants, flavorants and glidants. Examples of such ingredients include but are not limited to microcrystalline cellulose or calcium phosphate dibasic, calcium phosphate dihydrate, calcium sulfate dihydrate, cellulose derivatives, dextrose, lactose, anhydrous lactose, spray-dried lactose, lactose monohydrate, mannitol, starches, sorbitol and sucrose.

In some embodiments, these excipients may be present in varying amounts consistent with obtaining a suitable oral dosage form. In certain embodiments, excipients may be present in amounts of 1 to 99% by weight.

In some embodiments, a formulation contains lactose monohydrate in an amount of about 20% to about 40%; about 25% to about 35%; or about 27% to about 33%. In some embodiments, a formulation includes lactose monohydrate in an amount of about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; or about 40%. In some embodiments, a formulation includes lactose monohydrate in an amount of about 31.4%. In some embodiments, such percentages represent the amount of lactose monohydrate cellulose in a core layer of a formulation.

When a dosage form is intended to provide an immediate burst of drug substance after the lag time, the matrix may contain excipients commonly used in immediate release dosage forms.

In some embodiments, a matrix adapted for an immediate burst of drug substance upon expiry of the lag time may comprise a surface-active agent such as sodium lauryl sulfate, sodium monoglycerate, sorbitan monooleate, polyoxyethylene sorbitan monooleate, glyceryl monostearate, glyceryl monooleate, glyceryl monobutyrate, any one of the Pluronic line of surface-active polymers, or any other suitable material with surface active properties or any combination of the above. In some embodiments, surface active materials may be present in the dosage form in amounts of about 0.5% to about 10% by weight; about 1% to about 10% by weight; or about 3% to about 7% by weight. In some embodiments, surface active materials may be present in a dosage form in amounts of about 0.5% to about 10% by weight; about 1% to about 10% by weight; or about 3% to about 7% by weight. In some embodiments, surface active materials may be present in a dosage form in amounts of about 0.5% by weight; about 1% by weight; about 2% by weight; about 3% by weight; about 4% by weight; about 5% by weight; about 6% by weight; about 7% by weight; about 8% by weight; about 9% by weight; or about 10% by weight. In some embodiments, such percentages represent the amount of a surface active agent in a core layer of a formulation.

Other suitable ingredients commonly employed in immediate release formulations may include, but are not limited to, microcrystalline cellulose (such as Avicel), corn starch, pregelatinized starch (such as Starch 1500 or National 1551), potato starch, sodium carboxymethylated starch, sodium carboxymethylated cellulose, hydroxypropylmethyl cellulose (such as Methocel K100LV), hydroxypropylcellulose, hydroxyethylcellulose, and ethylcellulose. In addition, binder materials such as gums (e.g., guar gum) natural binders and derivatives such as alginates, chitosan, gelatin and gelatin derivatives, are also useful. Synthetic polymers such as polyvinylpyrrolidone (PVP), acrylic acid derivatives (Eudragit, Carbopol, etc.) and polyethylene glycol (PEG) are also useful as binders and matrix formers.

In some embodiments, a formulation includes hydroxypropylmethyl cellulose in an amount of about 20% to about 40%; about 25% to about 35%; or about 27% to about 33%. In some embodiments, a formulation includes hydroxypropylmethyl cellulose in an amount of about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; or about 40%. In some embodiments, a formulation includes hydroxypropylmethyl cellulose in an amount of about 31.4%. In some embodiments, such percentages represent the amount of hydroxypropylmethyl cellulose in a core layer of a formulation.

In some embodiments, polyvinyl pyrrolidone may be present in the dosage form in amounts of about 0.5% to about 10% by weight; about 1% to about 10% by weight; or about 3% to about 7% by weight. In some embodiments, polyvinyl pyrrolidone may be present in a dosage form in amounts of about 0.5% to about 10% by weight; about 1% to about 10% by weight; or about 3% to about 7% by weight. In some embodiments, polyvinyl pyrrolidone may be present in a dosage form in amounts of about 0.5% by weight; about 1% by weight; about 2% by weight; about 3% by weight; about 4% by weight; about 5% by weight; about 6% by weight; about 7% by weight; about 8% by weight; about 9% by weight; or about 10% by weight. In some embodiments, such percentages represent the amount of a surface active agent in a core layer of a formulation.

In some embodiments, it may also be desirable to incorporate a disintegrant into an immediate release matrix in order to facilitate dissolution of the drug substance. For this purpose, any suitable tablet disintegrant can be utilized here, such as cross-linked sodium carboxymethylcellulose (Ac-Di-Sol), cross-linked sodium carboxymethyl starch (Explotab, Primojel), cross-linked PVP (Plasdone XL) or any other material possessing tablet disintegrant properties. In some embodiments, such ingredients may be present in the dosage form in amounts of about 1% to about 99% by weight.

As will be immediately apparent to the skilled person, a wide variety of release profiles can be obtained having regard to the nature and composition of the core matrix. In some embodiments, the core may be of a multi-layered configuration, having both a release controlling layer and a layer for immediate release. In some embodiments, such layers are rendered distinct each from the other. This may be achieved by one layer including a colorant or a material that is opaque to x-rays, and the other not.

In some embodiments, dosage forms may be over-coated with a pharmaceutically acceptable film-coating, for aesthetic purposes (e.g. including a colorant), for stability purposes (e.g., coated with a moisture barrier), for taste-masking purposes, or for the purpose of protecting unstable drug substances from aggressive media, e.g. enteric-coatings.

Preparation of Dosage Forms

In some embodiments, dosage forms may take any suitable form, including capsules, tablets and pellets. Such dosage forms may be intended for administration by any known means, including oral, buccal and sublingual. In certain embodiments, the dosage form is adapted for oral delivery intended for ingestion. In some embodiments, the components of the dosage form comply with the U.S. Phatinacopeia (USP) General Chapter 467 requirement for control of residual solvents.

In some embodiments, dosage forms of the present invention may be prepared according to any of the techniques known in the art. Matrices may be formed by mixing release controlling agent, drug substance and any suitable tabletting excipients, including any of those materials referred to herein, and coated using techniques in the art.

For example, in some embodiments coatings may be formed by compression using any of the known press coaters. In some embodiments, dosage forms may be prepared by granulation and agglomeration techniques, or built up using spray drying techniques, followed by drying.

In some embodiments, coating thickness can be controlled precisely by employing any of the aforementioned techniques. The skilled person can select the coating thickness as a means to obtain a desired lag time, and/or the desired rate at which drug substance is released after the lag time.

For reasons of patient compliance, in some embodiments the dosage form is as small as possible and the coating has the minimum thickness possible consistent with achieving the desired lag time. In some embodiments, by the judicious selection of the coating materials, one is able to produce a coating that is relatively recalcitrant to the ingress of moisture and so long lag times can be achieved with relatively thin coatings.

In some embodiments, a dosage form is provided in the form of a press-coated tablet. In certain embodiments, the tablet comprises a core containing a drug substance, and a coating surrounding said core, the core being applied by press-coating coating material around a preformed core. In some embodiments, the coating may contain any of the release-controlling agents described herein.

In some embodiments, the coating comprises one or more water insoluble or poorly soluble hydrophobic excipients. In certain embodiments, these excipients are selected from fatty acids or their esters or salts; long chain fatty alcohols; polyoxyethylene alkyl ethers; polyoxyethylene stearates; sugar esters; lauroyl macrogol-32 glyceryl, stearoyl macrogol-32 glyceryl, and the like.

In some embodiments, other excipients that provide a hydrophobic quality to coatings may be selected from any waxy substance known for use as tablet excipients. In some embodiments, the excipients have a HLB value of less than about 5, or about 2. In some embodiments, suitable hydrophobic agents include waxy substances such as carnauba wax, paraffin, microcrystalline wax, beeswax, cetyl ester wax and the like; or non-fatty hydrophobic substances such as calcium phosphate salts, e.g. dibasic calcium phosphate.

In some embodiments, coatings comprising the aforementioned materials may provide for a lag time by acting as a barrier to the ingress of a physiological medium. Once the medium crosses the coating and enters the matrix causing the matrix to expand, for example by swelling, gelling or effervescing, the coating is broken open exposing the core matrix, thereby permitting release of drug substance from the matrix. In this way, in some embodiments the coating exerts no, or substantially no, influence over the release rate after expiry of the lag time.

In certain embodiments, coating ingredients include calcium phosphate salts, glyceryl behenate, and polyvinyl pyrollidone, or mixtures thereof, and one or more adjuvants, diluents, lubricants or fillers.

In some embodiments, a coating may include polyvinyl pyrollidone (Povidone) which may be present in amounts of about 1% to about 25% by weight of the coating, about 4% to about 12% by weight of the coating, or about 6% to about 8% by weight of the coating. In some embodiments, a coating may include polyvinyl pyrollidone in an amount of about 4% by weight; about 5% by weight; about 6% by weight; about 7% by weight; about 9% by weight; about 10% by weight; about 11% by weight; about 12% by weight; or about 6.53% by weight.

In some embodiments, a coating may include glyceryl behenate, an ester of glycerol and behenic acid (a C₂₂ fatty acid), which may be present as its mono-, di-, or tri-ester form, or a mixture thereof. In some embodiments, it has an HLB value of less than about 5, or about 2. In some embodiments, glyceryl behenate may be present in amounts of about 5% to about 85% by weight of the coating, about 10% to about 70% by weight of the coating, about 30% to about 50% by weight of the coating, about 10% to about 30% by weight of the coating; or about 15% to about 25% by weight of the coating. In some embodiments, glyceryl behenate may be present in amounts about 15% by weight of the coating; about 16% by weight of the coating; about 17% by weight of the coating; about 18% by weight of the coating; about 19% by weight of the coating; about 20% by weight of the coating; about 21% by weight of the coating; about 22% by weight of the coating; about 23% by weight of the coating; about 24% by weight of the coating; about 25% by weight of the coating; about 26% by weight of the coating; about 27% by weight of the coating; about 28% by weight of the coating; about 29% by weight of the coating; about 30% by weight of the coating; or about 21.1% by weight of the coating.

In some embodiments, a coating may include calcium phosphate salt, which may be the dibasic calcium phosphate dihydrate and which may be present in an amount of about 10% to about 90% by weight of the coating, about 20% to about 80% by weight of the coating, about 30% to about 50% by weight of the coating; or about 40% to about 75% by weight of the coating. In some embodiments, a coating may include calcium phosphate salt, which may be the dibasic calcium phosphate dihydrate and which may be present in an amount of about 30% by weight of the coating; about 31% by weight of the coating; about 32% by weight of the coating; about 33% by weight of the coating; about 34% by weight of the coating; about 35% by weight of the coating; about 36% by weight of the coating; about 37% by weight of the coating; about 38% by weight of the coating; about 39% by weight of the coating; about 34% by weight of the coating; about 41% by weight of the coating; about 42% by weight of the coating; about 43% by weight of the coating; about 44% by weight of the coating; about 45% by weight of the coating; about 46% by weight of the coating; about 47% by weight of the coating; about 48% by weight of the coating; about 49% by weight of the coating; about 50% by weight of the coating; or about 38.9% by weight of the coating.

In some embodiments, a coating may include microcrystalline cellulose in an amount of about 1% to about 50% by weight of the coating, about 1% to about 30% by weight of the coating, about 5% to about 20% by weight of the coating; or about 5% to about 15% by weight of the coating. In some embodiments, a coating may include microcrystalline cellulose in an amount of about 5% by weight of the coating; about 6% by weight of the coating; about 7% by weight of the coating; about 8% by weight of the coating; about 9% by weight of the coating; about 10% by weight of the coating; about 11% by weight of the coating; about 12% by weight of the coating; about 13% by weight of the coating; about 14% by weight of the coating; or about 15% by weight of the coating.

In some embodiments, the coating may contain other excipients commonly used in forming solid oral dosage forms, such as are described above. In some embodiments, press-coating provides a particularly effective means of controlling coating thickness, and therefore controlling the lag time. In some embodiments, press-coating is particularly advantageous as one can control coat weight, diameter of die and size of core to achieve a precisely defined minimum coating thickness at points on the dosage form. In some embodiments, ingress of a physiological medium across the coating at these points will determine the time period for the medium to reach the core and hydrate it, and the lag time may be controlled in this manner.

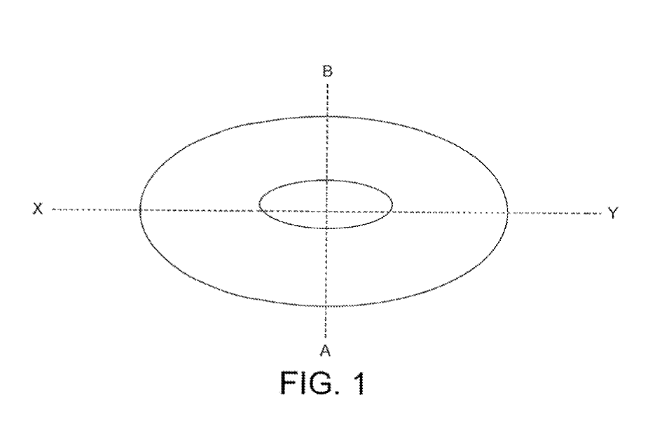

With reference to FIG. 1 below, the thickness of the coating along and about the axis of the direction of movement of a press-coater punch (the “A-B” axis) is determined by the amount of coating material added to the die and the compaction force applied to form of a dosage form. On the other hand, the thickness of the coating along and about the “X-Y” axis is determined by the size of the core, its position within the die and the diameter of the die in the press-coater. It will be apparent to the skilled person that even though FIG. 1 only shows a 2-dimensional representation of a dosage form, there is a plurality of axes X-Y orthogonal to the “A-B” axis, which extend radially from the centre of the dosage form to its circumference, and when the reference is made to the thickness of the coating about an axis X-Y, reference is being made the thickness about any or all of these axes.

Given that one can manipulate the thickness of the coating around or about the axis A-B to ensure it is thicker than the coating about the axis X-Y, ingress of moisture at X-Y will influence the lag time. Accordingly, the formulator has some latitude in selecting the thickness of the coating along A-B. It should not be so thick as to render the dosage form too large and therefore difficult to swallow, yet on the other hand it should not be so thin that the coating is render weak and liable to crack under the slightest mechanical stress.

In some embodiments, a dosage form comprises a press-coated tablet including a core and a coating surrounding the core, the coating having thickness about the axis X-Y such that upon immersion in an aqueous medium as described herein there will be less than about 10% release of drug substance, less than about 5%, less than about 2%, or less than about 1% during a lag time as defined herein above.

In some embodiments, the thickness of the coating about the axis X-Y may be about 2 to about 2.6 nm. The dosage form may be formed by compression coating methods as will be described in more detail herein below. In some embodiments, compression coated dosage forms may be formed by placing a portion of a powdered coating material in a die and tamping the powder into a compact form using a punch. A core may then be deposited onto the compacted coating material before the remainder of the coating material is introduced into the die and compression forces are applied to form the coated dosage form. To ensure that the core is placed on the tamped coating material and to ensure its correct geometry relative to the coating in the final tablet form, it may be preferable to employ means for positioning the core in relation to the coating material in a die. In some embodiments, such means may be provided by a pin punch having a convex surface that contacts the coating material to leave a small depression or hollow in the tamped coating material. Thus, when the core is placed into the die on the tamped material, it sits in the depression or hollow and its correct geometry is assured in the final tablet form.

As a result of this process, different areas of the formed tablet may experience different compaction forces, and therefore the coating may vary in density or porosity at different points. For example, the top portion of the coating along axis A-B (in the direction of the movement of the punch) is generally more compact compared with the bottom portion along the same axis. In an embodiment wherein the tablet core is multilayered, it is important to ensure that the cores are always the right way up along the A-B axis. A suitable detection device arranged in cooperation with a press coater can read whether the cores are in the correct position entering the press coater die, and reject those that are not, thus providing a means of in-process control. Using a colorant such as ferric oxide or excipients opaque to x-rays in a core containing only a single layer can also be advantageous to ensure that a core is correctly positioned with a coating. As an additional in-process control is achieved by means of a light or radiation detector suitably positioned in relation to the press-coater to inspect finished tablets to ensure that for a given dosage form, its core is correctly positioned within its coating.

During the compression of the coating around the core, the coating material above and below the core (the material along and about the A-B axis) is relatively highly compacted and dense. On the other hand, the coating material disposed along and about the X-Y axis may be subjected to lower compaction forces and may be relatively less dense. Accordingly, the material about the X-Y axis may be relatively porous and permissive towards the ingress of aqueous media. Because of the slightly less dense nature of the coating material along this axis, and because the formulator has the latitude to influence the coating thickness, in some embodiments the rate of ingress of the aqueous medium through the coating along the direction of the X-Y axis can be closely controlled.

Once an aqueous medium contacts the core, the core may react by swelling and/or gelling or effervescing thereby to break open the core generally along the direction of ingress of the aqueous media (i.e. the X-Y axis) to form to essentially two hemispheres of coating material that may remain conjoined. In this opened form, the dosage form may have the appearance of an opened shell. The reaction of the core material to the presence of the aqueous medium is in some embodiments likewise in part responsible for controlling the release of drug substance from the core.

In some embodiments, the hardness of the dosage form may be at least about 60 Newtons, e.g. 60 to 80 Newtons, and more particularly 60 to 75 Newtons. Hardness may be measured according to a process described in The European Pharmacopoeia 4, 2.9.8 at page 201. The test employs apparatus consisting of 2 opposing jaws, one of which moves toward the other. The flat surfaces of the jaws are perpendicular to the direction of movement. The crushing surfaces of the jaws are flat and larger than the zone of contact with the dosage form. The apparatus is calibrated using a system with a precision of one Newton. The dosage form is placed between the jaws. For each measurement, the dosage form is oriented in the same way with respect to the direction of the applied force. Measurements are carried out on 10 tablets. Results are expressed in terms of the mean, minimum and maximum values (in Newtons) of the force needed to crush the dosage form. Dosage forms having a hardness within this range are mechanically robust to withstand forces generated in the stomach, particularly in the presence of food. Furthermore, the dosage forms are sufficiently porous about the X-Y plane of the tablet to permit ingress of physiological media to the core at an appropriate rate to ensure lag times referred to herein above.

The invention provides in another aspect, a method of forming press-coated dosage forms as herein above described. They may be formed on conventional press coating equipment. Typically such equipment is composed of a series of die are arranged on a rotating platform. The die are removably mounted in the platform such that differently sized die may be employed as appropriate. Each die is hollow to receive a lower punch. The punch is positioned within the die such that the upper surface of the punch and the inner surface of the die define a volume for receiving a precise amount coating material. Once loaded, the platform is rotated until the die is positioned under an upper punch. The upper punch is then urged down onto the coating material under a defined compression force and the coating material is precompressed or tamped between the upper and lower punch. A pre-formed core is then fed into die to rest on the tamped coating. Conventional press coating apparatus may be equipped with centering devices that enable cores to be positioned both vertically and radially. This might be achieved by a tamping process, whereby an initial amount of coating material is placed in a die and is tamped with a shaped punch, such as a pin punch, that leaves an indentation in the coating material in which to receive a core. Thereafter, in a second filling operation, a precise amount of coating material is fed into the die to cover the core, and an upper punch compresses the coating material with a defined compaction force to form press-coated dosage forms.

The compression force applied during the tamping process is relatively light and is just sufficient to provide a bed of coating material to receive the core and to prevent movement of the coating material as a result of centrifugal force. Subsequent compression to form the dosage form may be adjusted to give a requisite hardness. In some embodiments, this compression force is 400 kg, although this may be adjusted by ±30% in order to give tablets of the required hardness.

The amount of coating material fed into the die can be precisely defined having regard to the density of the coating material to ensure after compression that the dosage form is formed with the required coating thickness about the A-B axis; and the dimensions of the die is selected to provide the thickness about the X-Y axis. Should it be necessary to change the thickness of the coating, die of appropriate internal dimensions may be placed in the rotating platform, and the amount of coating material fed into the die may be adjusted accordingly. Suitable rotary tablet machines having high process speeds are known in the art.

Cores may likewise be formed using a conventional rotary tablet machine. Cores may be compressed under compression forces sufficient to provide cores having a hardness of about 60 Newtons at least, e.g. 50 to 70 Newtons. Cores having hardness in this range give desired release characteristics. If desired, the cores can be formed at the same time as the press coated tablets are produced. In such case, one might employ a Manesty Dry Cota. Such a press consists of two side-by-side and inter-connected presses where the core is made on one press before being mechanically transferred to the other press for compression coating. Such equipment and techniques for making dosage forms using such equipment are known in the art and no more needs to be said about this here.

In some embodiments, cores are formed according to wet granulation techniques generally known in the art. In a typical procedure, core materials are sieved and blended. Granulating fluid, typically water is then added to the blend and the mixture is homogenized to form a granulate, which is then sprayed dried or dried on a fluid bed drier to obtain a granulate with requisite residual moisture. In some embodiments, the residual moisture content is from about 0.4% to about 2.0% by weight. The granulate is then sized by passing it through screens of desired aperture. At this stage, any adjuvants are sized and added to the granulate to form the core composition suitable for compression. The skilled person will appreciate that a coating composition can be formed in an analogous manner.

The skilled person will also appreciate that granulates may be obtained having a range of particle sizes. In some embodiments, the coating granulate has a fine fraction that is less than 30%. By “fine fraction” is meant granulate having particle size of up to about 63 microns.

As used herein, the term “about” is understood to mean±10% of the value referenced. For example, “about 10%” is understood to literally mean 9% to 11%.

A number of references have been cited, the entire disclosures of which are incorporated herein by reference.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

A core containing drug substance is prepared for the press coated system as follows. The composition of the core is detailed in Table 1. Lactose monohydrate (Lactose Pulvis.H₂O®, Danone, France and Lactose Fast Flo® NF 316, Foremost Ing. Group, USA) is a filling agent with interesting technical and functional properties. Lactose Pulvis.H₂O® is used in a blend prepared by wet granulation and Lactose Fast Flo is used in a blend prepared for direct compression. Microcrystalline cellulose (Avicel® pH 101, FMC International, Ireland) is used as an insoluble diluent for direct compression. Polyvinyl pyrrolidone (Plasdone® K29-32, ISP Technology, USA) is a granulating agent, soluble in water, which has the ability of binding the powder particles. Croscarmellose sodium (Ac-Di-Sol®, FMC Corporation, USA) is used in the formulation as a super disintegrant. As the external phase, magnesium stearate (Merck, Switzerland) was added as a lubricant and silicon dioxide (Aerosil® 200, Degussa AG, Germany) in order to improve flow properties of the granular powder.

TABLE 1

| Ingredients | Content (mg/tablet) |
|---|---|
| Drug Substance A | 5.00 |
| Lactose (Lactose Pulvis H₂O NF 316) | 39.10 |
| Polyvinyl pyrrolidone (Plasdone ® K29-32) | 4.00 |
| Sodium carboxymethyl cellulose (Ac-Di-Sol ®) | 11.00 |
| Magnesium stearate | 0.60 |
| Silicon dioxide (Aerosil ® 200) | 0.30 |
| Total | 60.00 |

The coating material is of a hydrophobic, water insoluble nature. This coating is composed of dibasic calcium phosphate (Emcompress®, Mendell, USA) and glyceryl behenate (Compritol® 888ATO, Gattefossé, France). Polyvinylpyrrolidone (Plasdone® K29-32) is a granulating agent, soluble in water, which has the ability of binding the powder particles. Yellow ferric oxide (Sicovit® Yellow 10, BASF, Germany) was added as a dye. A detailed composition of this barrier blend is given in table 2.

TABLE 2
Composition of the coating

| Ingredients | Content (%) |
|---|---|
| Dibasic calcium phosphate (Emcompress ®) | 50.00 |
| Glyceryl Behenate (Compritol ® 888 ATO) | 40.00 |
| Polyvinylpyrrolidone (Plasdone ® K29-32) | 8.40 |
| Yellow Ferric Oxide (Sicovit ® yellow 10 E 172) | 0.10 |
| Silicon dioxide (Aerosil ® 200) | 0.50 |
| Magnesium stearate | 1.00 |
| Total | 100.00 |

The required amounts of drug substance A, Ac-Di-Sol®, Lactose Pulvis H₂O, Plasdone® K29-32 were weighed and manually sieved with a screen having 0.710 mm apertures. The components were homogeneously mixed in a Niro-Fielder PMA 25-liter mixing granulator for 6 min at impeller speed 250 rpm without chopper. Subsequently, the granulating solution (purified water, 25.47% of the weight of the dry blend) was added within 4 min at impeller speed 250 rpm and chopper speed 1500 rpm, using a nozzle H1/4VV-95015 (spraying rate of 250 g/min). Mixing was continued for homogenization and massing of the wet mass for 3 min at impeller speed 500 rpm and chopper speed 3000 rpm.

The mixed wet granulate is then dried in a Glatt WSG5 fluidized air bed drier. The inlet temperature is maintained at 45° C. during drying. The drying lasted 20 min to obtain a granulate with a residual moisture less than 2.5%. The yielded dry granulate is calibrated in a Frewitt MGI 205 granulator using a screen with 0.8 mm apertures for 3 min at speed 244 osc/min (graduation 7). Appropriate amounts of Aerosil® 200 and magnesium stearate are manually sieved using a screen with 1.0 mm apertures. Half of the dry granulate is put in a Niro-Fielder PMA 25-liter mixing granulator, followed by Aerosil® 200 and then by the other half of the dry granulate. The ingredients are mixed for 2 min at impeller speed 250 rpm. Finally, magnesium stearate is added and mixing is continued for 2 min at impeller speed 250 rpm.

The coating blend is prepared according to the process described below. Batch size for the barrier blend is 13 kg. Weighed amounts of Encompress®, Compritol® 888 ATO, Lactose pulvis.H₂O®, Plasdone® K29-32 and Sicovit® Yellow 10 E 172 are manually sieved with a screen having 0.710 mm apertures. They are placed in a Niro-Fielder PMA 65-liter mixing granulator. Then, the components are homogeneously mixed for 6 min, at impeller speed 200 rpm, without chopper. Subsequently, the granulating solution (purified water, 8.12% of the weight of the dry blend) is added within 2 min at impeller speed 200 rpm and chopper speed 1500 rpm using a nozzle 4,9 (spraying rate of 520 g/min). Mixing is continued for homogenisation and massing for 1 min at impeller speed 400 rpm and chopper speed 3000 rpm.

The mixed wet granulate is then dried in a Niro-Fielder TSG 2 fluidised air bed dryer. The inlet temperature is maintained at 45° C. during drying. The drying lasted 33 min to have residual moisture less than 2.5%. The yielded dry granulate is calibrated in a Frewitt MGI 205 granulator using a screen having 0.8 mm apertures for 4 min at speed 244 osc/min (graduation 7). Appropriate amounts of Aerosil® 200 and magnesium stearate are manually sieved using a screen with 1.0 mm apertures. Half of the dry granulate is put in a Niro-Fielder PMA 65-liter, followed by Aerosil® 200 and then by the other half of the dry granulate. The ingredients are mixed for 2 min at impeller speed 200 rpm, without chopper. Finally, magnesium stearate is added and mixing is continued for 2 more minutes at impeller speed 200 rpm, without chopper.

440 mg of coating blend is press coated on a core to provide press coated tablets (9 mm diameter). 305 mg of coating blend is press coated on a core to provide press coated tablets (8 mm diameter). These different press coatings are made utilizing a Kilian RUD tabletting machine. First and second loading hoppers are filled up with the coating granulate. Between the two loading hoppers, the machine is equipped with a transfer system adapted to feed the cores. For each tablet, the first loading hopper supplies with about half of the quantity to be applied to the core. Then, the feeding system provides and positions a core centered in the die. Subsequently, the second loading hopper supplies with the other half of the quantity to be applied to the core. The compression step then occurs.

### Example 2

The in vitro dissolution profile of a tablet containing a 5 mg loading of drug substance A prepared according to the method of Example 1 is determined using USP dissolution apparatus No. 2 (paddles) and stationary baskets and applying a stirring rate of 100 rpm. The dissolution medium was purified water, with a volume of 1000 ml.

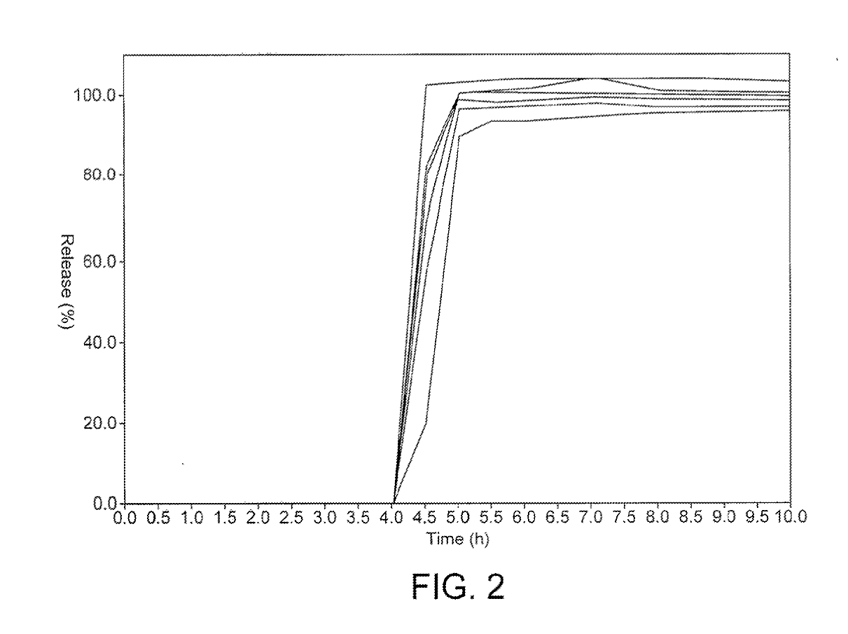

FIG. 2 shows the release profiles of several tablets formed according to the above formulation and methodology. The figure clearly shows that it is possible to obtain lag times with a very high degree of precision.

### Example 3

### Formulation 53Q1 (1 Hour Time Lag, 4 Hour Sustained Release)

A core containing drug substance is prepared for the press coated system as follows. The composition of the core is detailed in Table 3. Lactose monohydrate (Lactose Pulvis.H₂O®, Danone, France and Lactose Fast Flo® NF 316, Foremost Ing. Group, USA) is a filling agent with interesting technical and functional properties. Lactose Pulvis.H₂O is used in a blend prepared by wet granulation and Lactose Fast Flo is used in a blend prepared for direct compression. Hydroxypropylmethyl cellulose (Methocel K4M) is used to modify the release of the active agent (Zaleplon). Polyvinyl pyrrolidone (Plasdone® K-29-32, ISP Technology, USA) is a granulating agent, soluble in water, which has the ability of binding the powder particles. Sodium lauryl sulphate is a surfactant which helps to wet or hydrate the core and may help to solubilize the active agent. Red ferric oxide is added as a visual indicator to assist in ensuring that the core is correctly centered in the tablet punch. As the external phase, magnesium stearate (Merck, Switzerland) was added as a lubricant and silicon dioxide (Aerosil® 200, Degussa AG, Germany) in order to improve flow properties of the granular powder.

TABLE 3
Formulation of the core 1041/32E1 made with 1041/21 SR1

| Ingredients | Content (mg/tablet) | % |
|---|---|---|
| Zaleplon | 15.00 | 25.00 |
| Lactose (Lactose Pulvis | 11.00 | 18.33 |
| H₂O NF 316) | | |
| Polyvinyl pyrrolidone | 3.00 | 5.00 |
| (Plasdone ® K29-32) | | |
| Methocel K4M hydroxypropylmethyl | 22.00 | 36.67 |
| cellulose) | | |
| Magnesium stearate | 1.00 | 1.67 |
| Silicon dioxide (Aerosil ® 200) | 0.60 | 1.00 |
| Sodium lauryl sulphate | 7.00 | 11.67 |
| Red ferric oxide | 0.40 | 0.67 |
| Total | 60.00 | 100.00 |

The coating material is of a hydrophobic, water insoluble nature. This coating is composed of dibasic calcium phosphate dihydrate (Calipharm®, CAS 7789-77-7) and glyceryl behenate (Compritol® 888ATO, Gattefossé, France). Polyvinylpyrrolidone (Plasdone® K29-32) is a granulating agent, soluble in water, which has the ability of binding the powder particles. Yellow ferric oxide (Sicovit® Yellow 10, BASF, Germany) was added as a dye. Xylitol 300 (Xylisorb, CAS 87-99-0) is used as a hydrophilic compound, while sodium lauryl sulphate (CAS 151-21-3) is added as a hydrophilic compound and solubilizing agent.

A detailed composition of this barrier blend is given in table 4.

TABLE 4
Composition of the coating

| Ingredients | mg/tab | Content (%) |
|---|---|---|
| Dibasic calcium phosphate dihydrate | 145.75 | 32.75 |
| (Calipharm ®, CAS 7789-77-7) | | |
| Glyceryl Behenate (Compritol ® 888 ATO) | 116.60 | 26.20 |
| Xylitol 300 (Xylisorb, CAS 87-99-0) | 133.50 | 30.00 |
| Sodium lauryl sulphate (CAS 151-21-3) | 20.00 | 4.49 |
| Polyvinylpyrrolidone (Plasdone ® K29-32) | 24.49 | 5.50 |
| Yellow Ferric Oxide (Sicovit ® yellow 10 E | 0.29 | 0.07 |
| 172) | | |
| Silicon dioxide (Aerosil ® 200) | 1.46 | 0.33 |
| Magnesium stearate | 2.92 | 0.66 |
| Total | 445.00 | 100.00 |

The required amounts of Zaleplon, Methocel K4M, Lactose Pulvis H₂O®, Plasdone® K29-32 were weighed and manually sieved with a screen having 0.710 mm apertures. The components were homogeneously mixed in a Niro-Fielder PMA 25-liter mixing granulator for 6 min at impeller speed 250 rpm without chopper. Subsequently, the granulating solution (purified water, 25.47% of the weight of the dry blend) was added within 4 min at impeller speed 250 rpm and chopper speed 1500 rpm, using a nozzle H1/4VV-95015 (spraying rate of 250 g/min). Mixing was continued for homogenization and massing of the wet mass for 3 min at impeller speed 500 rpm and chopper speed 3000 rpm.

The mixed wet granulate is then dried in a Glatt WSG5 fluidized air bed drier. The inlet temperature is maintained at 45° C. during drying. The drying lasted 20 min to obtain a granulate with a residual moisture less than 2.5%. The yielded dry granulate is calibrated in a Frewitt MGI 205 granulator using a screen with 0.8 mm apertures for 3 min at speed 244 osc/min (graduation 7). Appropriate amounts of Aerosil® 200 and magnesium stearate are manually sieved using a screen with 1.0 mm apertures. Half of the dry granulate is put in a Niro-Fielder PMA 25-liter mixing granulator, followed by Aerosil® 200 and then by the other half of the dry granulate. The ingredients are mixed for 2 min at impeller speed 250 rpm. Finally, magnesium stearate is added and mixing is continued for 2 min at impeller speed 250 rpm.

The coating blend is prepared according to the process described below. Batch size for the barrier blend is 13 kg. Weighed amounts of Calipharm®, Compritol® 888 ATO, Lactose pulvis.H₂O®, Plasdone® K29-32 and Sicovit® Yellow 10 E 172 are manually sieved with a screen having 0.710 mm apertures. They are placed in a Niro-Fielder PMA 65-liter mixing granulator. Then, the components are homogeneously mixed for 6 min, at impeller speed 200 rpm, without chopper. Subsequently, the granulating solution (purified water, 8.12% of the weight of the dry blend) is added within 2 min at impeller speed 200 rpm and chopper speed 1500 rpm using a nozzle 4,9 (spraying rate of 520 g/min). Mixing is continued for homogenization and massing for 1 min at impeller speed 400 rpm and chopper speed 3000 rpm.

The mixed wet granulate is then dried in a Niro-Fielder TSG 2 fluidized air bed dryer. The inlet temperature is maintained at 45° C. during drying. The drying lasted 33 min to have residual moisture less than 2.5%. The yielded dry granulate is calibrated in a Frewitt MGI 205 granulator using a screen having 0.8 mm apertures for 4 min at speed 244 osc/min (graduation 7). Appropriate amounts of Aerosil® 200 and magnesium stearate are manually sieved using a screen with 1.0 mm apertures. Half of the dry granulate is put in a Niro-Fielder PMA 65-liter, followed by Aerosil® 200 and then by the other half of the dry granulate. The ingredients are mixed for 2 min at impeller speed 200 rpm, without chopper. Finally, magnesium stearate is added and mixing is continued for 2 more minutes at impeller speed 200 rpm, without chopper.

440 mg of coating blend is press coated on a core to provide press coated tablets (9 mm diameter). 305 mg of coating blend is press coated on a core to provide press coated tablets (8 mm diameter). These different press coatings are made utilizing a Kilian RUD tabletting machine. First and second loading hoppers are filled up with the coating granulate. Between the two loading hoppers, the machine is equipped with a transfer system adapted to feed the cores. For each tablet, the first loading hopper supplies with about half of the quantity to be applied to the core. Then, the feeding system provides and positions a core centered in the die. Subsequently, the second loading hopper supplies with the other half of the quantity to be applied to the core. The compression step then occurs.

### Example 4

### Formulation 51Q1 (2 Hour Time Lag Immediate Release)

A core containing drug substance is prepared for the press coated system as follows. The composition of the core is detailed in Table 5. Lactose monohydrate (Lactose Pulvis.H₂O®, Danone, France and Lactose Fast Flo® NF 316, Foremost Ing. Group, USA) is a filling agent with interesting technical and functional properties. Lactose Pulvis.H₂O® is used in a blend prepared by wet granulation and Lactose Fast Flo is used in a blend prepared for direct compression. Croscarmellose sodium (Ac-Di-Sol, FMC Corporation, USA) is used in the formulation as a super disintegrant. Polyvinyl pyrrolidone (Plasdone® K29-32, ISP Technology, USA) is a granulating agent, soluble in water, which has the ability of binding the powder particles. Sodium lauryl sulphate is a surfactant which helps to wet or hydrate the core and may help to solubilize the active agent. Red ferric oxide is added as a visual indicator to assist in ensuring that the core is correctly centered in the tablet punch. As the external phase, magnesium stearate (Merck, Switzerland) was added as a lubricant and silicon dioxide (Aerosil® 200, Degussa AG, Germany) in order to improve flow properties of the granular powder.

TABLE 5
Formulation of the core 1041/29E1 made with 1041/02FR1

| Ingredients | Content (mg/tablet) | % |
|---|---|---|
| Zaleplon | 15.00 | 25.00 |
| Lactose (Lactose Pulvis H₂O NF | 25.80 | 43.00 |
| 316) | | |
| Polyvinyl pyrrolidone (Plasdone ® | 4.00 | 6.67 |
| K29-32) | | |
| Sodium carboxymethyl cellulose | 11.00 | 18.33 |
| (Ac-Di-Sol ®) | | |
| Magnesium stearate | 0.60 | 1.00 |
| Silicon dioxide (Aerosil ® 200) | 0.30 | 0.50 |
| Sodium lauryl sulphate | 3.00 | 5.00 |
| Red ferric oxide | 0.30 | 0.50 |
| Total | 60.00 | 100.00 |

The coating material is of a hydrophobic, water insoluble nature. This coating is composed of dibasic calcium phosphate dihydrate (Calipharm®, CAS 7789-77-7) and glyceryl behenate (Compritol® 888ATO, Gattefosse, France). Polyvinylpyrrolidone (Plasdone® K29-32) is a granulating agent, soluble in water, which has the ability of binding the powder particles. Yellow ferric oxide (Sicovit® Yellow 10, BASF, Germany) was added as a dye. Xylitol 300 (Xylisorb, CAS 87-99-0) is used as a hydrophilic compound, while sodium lauryl sulphate (CAS 151-21-3) is added as a hydrophilic compound and solubilizing agent.

A detailed composition of this barrier blend is given in table 6.

TABLE 6
Composition of the coating

| Ingredients | mg/tab | Content (%) |
|---|---|---|
| Dibasic calcium phosphate dihydrate | 173.00 | 38.88 |
| (Calipharm ®, CAS 7789-77-7) | | |
| Glyceryl Behenate (Compritol ® 888 ATO) | 138.40 | 31.10 |
| Xylitol 300 (Xylisorb, CAS 87-99-0) | 89.00 | 20.00 |
| Sodium lauryl sulphate (CAS 151-21-3) | 10.00 | 2.25 |
| Polyvinylpyrrolidone (Plasdone ® K29-32) | 29.06 | 6.53 |
| Yellow Ferric Oxide (Sicovit ® yellow 10 | 0.35 | 0.08 |
| E 172) | | |
| Silicon dioxide (Aerosil ® 200) | 1.73 | 0.39 |
| Magnesium stearate | 3.46 | 0.78 |
| Total | 445.00 | 100.00 |

The required amounts of Zaleplon, Methocel K4M, Lactose Pulvis H₂O®, Plasdone® K29-32 were weighed and manually sieved with a screen having 0.710 mm apertures. The components were homogeneously mixed in a Niro-Fielder PMA 25-liter mixing granulator for 6 min at impeller speed 250 rpm without chopper. Subsequently, the granulating solution (purified water, 25.47% of the weight of the dry blend) was added within 4 min at impeller speed 250 rpm and chopper speed 1500 rpm, using a nozzle H1/4VV-95015 (spraying rate of 250 g/min). Mixing was continued for homogenization and massing of the wet mass for 3 min at impeller speed 500 rpm and chopper speed 3000 rpm.

The mixed wet granulate is then dried in a Glatt WSG5 fluidized air bed drier. The inlet temperature is maintained at 45° C. during drying. The drying lasted 20 min to obtain a granulate with a residual moisture less than 2.5%. The yielded dry granulate is calibrated in a Frewitt MGI 205 granulator using a screen with 0.8 mm apertures for 3 min at speed 244 osc/min (graduation 7). Appropriate amounts of Aerosil® 200 and magnesium stearate are manually sieved using a screen with 1.0 mm apertures. Half of the dry granulate is put in a Niro-Fielder PMA 25-liter mixing granulator, followed by Aerosil® 200 and then by the other half of the dry granulate. The ingredients are mixed for 2 min at impeller speed 250 rpm. Finally, magnesium stearate is added and mixing is continued for 2 min at impeller speed 250 rpm.

The coating blend is prepared according to the process described below. Batch size for the barrier blend is 13 kg. Weighed amounts of Calipharm®, Compritol® 888 ATO, Lactose pulvis H₂O®, Plasdone® K29-32 and Sicovit® Yellow 10 E 172 are manually sieved with a screen having 0.710 mm apertures. They are placed in a Niro-Fielder PMA 65-liter mixing granulator. Then, the components are homogeneously mixed for 6 min, at impeller speed 200 rpm, without chopper. Subsequently, the granulating solution (purified water, 8.12% of the weight of the dry blend) is added within 2 min at impeller speed 200 rpm and chopper speed 1500 rpm using a nozzle 4,9 (spraying rate of 520 g/min). Mixing is continued for homogenisation and massing for 1 min at impeller speed 400 rpm and chopper speed 3000 rpm.

The mixed wet granulate is then dried in a Niro-Fielder TSG 2 fluidized air bed dryer. The inlet temperature is maintained at 45° C. during drying. The drying lasted 33 min to have residual moisture less than 2.5%. The yielded dry granulate is calibrated in a Frewitt MGI 205 granulator using a screen having 0.8 mm apertures for 4 min at speed 244 osc/min (graduation 7). Appropriate amounts of Aerosil® 200 and magnesium stearate are manually sieved using a screen with 1.0 mm apertures. Half of the dry granulate is put in a Niro-Fielder PMA 65-liter, followed by Aerosil® 200 and then by the other half of the dry granulate. The ingredients are mixed for 2 min at impeller speed 200 rpm, without chopper. Finally, magnesium stearate is added and mixing is continued for 2 more minutes at impeller speed 200 rpm, without chopper.

440 mg of coating blend is press coated on a core to provide press coated tablets (9 mm diameter). 305 mg of coating blend is press coated on a core to provide press coated tablets (8 mm diameter). These different press coatings are made utilizing a Kilian RUD tabletting machine. First and second loading hoppers are filled up with the coating granulate. Between the two loading hoppers, the machine is equipped with a transfer system adapted to feed the cores. For each tablet, the first loading hopper supplies with about half of the quantity to be applied to the core. Then, the feeding system provides and positions a core centered in the die. Subsequently, the second loading hopper supplies with the other half of the quantity to be applied to the core. The compression step then occurs.

### Example 5

### Formulation 54Q1 (2 Hour Time Lag, 2 Hour Sustained Release)

A core containing drug substance is prepared for the press coated system as follows. The composition of the core is detailed in Table 7. Lactose monohydrate (Lactose Pulvis H₂O®, Danone, France and Lactose Fast Flo® NF 316, Foremost Ing. Group, USA) is a filling agent with interesting technical and functional properties. Lactose Pulvis H₂O is used in a blend prepared by wet granulation and Lactose Fast Flo is used in a blend prepared for direct compression. Hydroxypropylmethyl cellulose (Methocel K100LV) is used to modify the release of the active agent (Zaleplon). Polyvinyl pyrrolidone (Plasdone® K29-32, ISP Technology, USA) is a granulating agent, soluble in water, which has the ability of binding the powder particles. Sodium lauryl sulphate is a surfactant which helps to wet or hydrate the core and may help to solubilize the active agent. Red ferric oxide is added as a visual indicator to assist in ensuring that the core is correctly centered in the tablet punch. As the external phase, magnesium stearate (Merck, Switzerland) was added as a lubricant and silicon dioxide (Aerosil® 200, Degussa AG, Germany) in order to improve flow properties of the granular powder.

TABLE 7
Formulation of the core 1041/33E1 made with 1 041/22SR1

| Ingredients | Content (mg/tablet) | % |
|---|---|---|
| Zaleplon | 15.00 | 25.00 |
| Lactose (Lactose Pulvis H₂O NF 316) | 11.00 | 18.33 |
| Polyvinyl pyrrolidone (Plasdone ® | 3.00 | 5.00 |
| K29-32) | | |
| Methocel K4M | 22.00 | 36.67 |
| (hydroxypropylmethyl cellulose) | | |
| Magnesium stearate | 1.00 | 1.67 |
| Silicon dioxide (Aerosil ® 200) | 0.60 | 1.00 |
| Sodium lauryl sulphate | 7.00 | 11.67 |
| Red ferric oxide | 0.40 | 0.67 |
| Total | 60.00 | 100.00 |

The coating material is of a hydrophobic, water insoluble nature. This coating is composed of dibasic calcium phosphate dihydrate (Calipharm®, CAS 7789-77-7) and glyceryl behenate (Compritol® 888ATO, Gattefosse, France). Polyvinylpyrrolidone (Plasdone® K29-32) is a granulating agent, soluble in water, which has the ability of binding the powder particles. Yellow ferric oxide (Sicovit® Yellow 10, BASF, Germany) was added as a dye. Xylitol 300 (Xylisorb, CAS 87-99-0) is used as a hydrophilic compound, while sodium lauryl sulphate (CAS 151-21-3) is added as a hydrophilic compound and solubilizing agent.

A detailed composition of this barrier blend is given in table 8.

TABLE 8
Composition of the coating

| Ingredients | mg/tab | Content (%) |
|---|---|---|
| Dibasic calcium phosphate dihydrate | 173.00 | 38.88 |
| (Calipharm ®, CAS 7789-77-7) | | |
| Glyceryl Behenate (Compritol ® 888 ATO) | 138.40 | 31.10 |
| Xylitol 300 (Xylisorb, CAS 87-99-0) | 89.00 | 20.00 |
| Sodium lauryl sulphate (CAS 151-21-3) | 10.00 | 2.25 |
| Polyvinylpyrrolidone (Plasdone ® K29-32) | 29.06 | 6.53 |
| Yellow Ferric Oxide (Sicovit ® yellow 10 E 172) | 0.35 | 0.08 |
| Silicon dioxide (Aerosil ® 200) | 1.73 | 0.39 |
| Magnesium stearate | 3.46 | 0.78 |
| Total | 445.00 | 100.00 |

The required amounts of Zaleplon, Methocel K4M, Lactose Pulvis H₂O®, Plasdone® K29-32 were weighed and manually sieved with a screen having 0.710 mm apertures. The components were homogeneously mixed in a Niro-Fielder PMA 25-liter mixing granulator for 6 min at impeller speed 250 rpm without chopper. Subsequently, the granulating solution (purified water, 25.47% of the weight of the dry blend) was added within 4 min at impeller speed 250 rpm and chopper speed 1500 rpm, using a nozzle H1/4VV-95015 (spraying rate of 250 g/min). Mixing was continued for homogenization and massing of the wet mass for 3 min at impeller speed 500 rpm and chopper speed 3000 rpm.

The mixed wet granulate is then dried in a Glatt WSG5 fluidised air bed drier. The inlet temperature is maintained at 45° C. during drying. The drying lasted 20 min to obtain a granulate with a residual moisture less than 2.5%. The yielded dry granulate is calibrated in a Frewitt MGI 205 granulator using a screen with 0.8 mm apertures for 3 min at speed 244 osc/min (graduation 7). Appropriate amounts of Aerosil® 200 and magnesium stearate are manually sieved using a screen with 1.0 mm apertures. Half of the dry granulate is put in a Niro-Fielder PMA 25-liter mixing granulator, followed by Aerosil® 200 and then by the other half of the dry granulate. The ingredients are mixed for 2 min at impeller speed 250 rpm. Finally, magnesium stearate is added and mixing is continued for 2 min at impeller speed 250 rpm.

The coating blend is prepared according to the process described below. Batch size for the barrier blend is 13 kg. Weighed amounts of Calipharm®, Compritol® 888 ATO, Lactose pulvis H2O®, Plasdone® K29-32 and Sicovit® Yellow 10 E 172 are manually sieved with a screen having 0.710 mm apertures. They are placed in a Niro-Fielder PMA 65-liter mixing granulator. Then, the components are homogeneously mixed for 6 min, at impeller speed 200 rpm, without chopper. Subsequently, the granulating solution (purified water, 8.12% of the weight of the dry blend) is added within 2 min at impeller speed 200 rpm and chopper speed 1500 rpm using a nozzle 4,9 (spraying rate of 520 g/min). Mixing is continued for homogenization and massing for 1 min at impeller speed 400 rpm and chopper speed 3000 rpm.

The mixed wet granulate is then dried in a Niro-Fielder TSG 2 fluidised air bed dryer. The inlet temperature is maintained at 45° C. during drying. The drying lasted 33 min to have residual moisture less than 2.5%. The yielded dry granulate is calibrated in a Frewitt MGI 205 granulator using a screen having 0.8 mm apertures for 4 min at speed 244 osc/min (graduation 7). Appropriate amounts of Aerosil® 200 and magnesium stearate are manually sieved using a screen with 1.0 mm apertures. Half of the dry granulate is put in a Niro-Fielder PMA 65-liter, followed by Aerosil® 200 and then by the other half of the dry granulate. The ingredients are mixed for 2 min at impeller speed 200 rpm, without chopper. Finally, magnesium stearate is added and mixing is continued for 2 more minutes at impeller speed 200 rpm, without chopper.

440 mg of coating blend is press coated on a core to provide press coated tablets (9 mm diameter). 305 mg of coating blend is press coated on a core to provide press coated tablets (8 mm diameter). These different press coatings are made utilizing a Kilian RUD tabletting machine. First and second loading hoppers are filled up with the coating granulate. Between the two loading hoppers, the machine is equipped with a transfer system adapted to feed the cores. For each tablet, the first loading hopper supplies with about half of the quantity to be applied to the core. Then, the feeding system provides and positions a core centered in the die. Subsequently, the second loading hopper supplies with the other half of the quantity to be applied to the core. The compression step then occurs.

### Example 6

The in vitro dissolution profile of tablets each containing a 5 mg loading of Zaleplon prepared according to the method of Examples 3, 4 and 5 respectively is determined using USP dissolution apparatus No. 2 (paddles) and stationary baskets and applying a stirring rate of 100 rpm. The dissolution medium was 0.02% sodium lauryl sulphate in 500 ml distilled water, with a volume of 1000 ml.

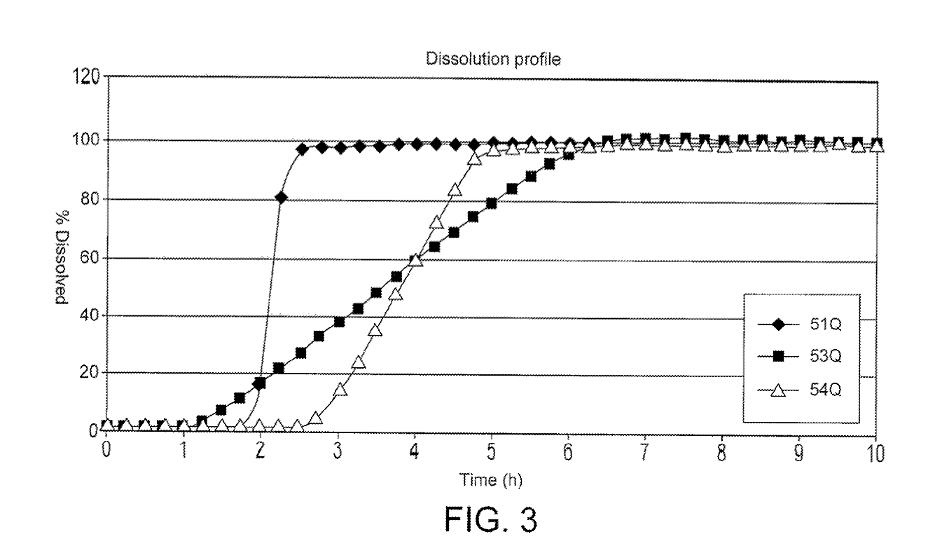

FIG. 3 illustrates the release of Zaleplon from the formulations of Examples 3-5. A lag time of at least one hour is observed in each case, followed by immediate release (Example 4) or delayed release (Examples 3 and 5) of the active agent.

### Example 7

A dosage form was prepared according to the formulation in Table 9:

TABLE 9

| Content | % |
|---|---|
| Blend 1 (Core) | |
| Internal Phase | |
| Zaleplon | 25.0 |
| Methocel K100LV | 31.4 |
| (hydroxypropylmethyl cellulose) | |
| Lactose pulvis, H₂O | 31.4 |
| (lactose monohydrate) | |
| SLS | 5.00 |
| Plasdone K29-32 (PVP) | 5.00 |
| Sicovit Red 30 E 172 | 0.67 |
| External Phase | |
| Aerosil 200 (silicon dioxide) | 1.00 |
| Magnesium stearate | 0.50 |
| Total, layer | 100 |
| Blend 2 (Shell) | |
| Internal Phase | |
| Dibasic calcium phosphate, 2H₂O | 38.9 |
| Compritol 888 ATO | 21.1 |
| (glyceryl behenate) | |
| Xylitol 300 | 20.0 |
| Avicel PH101 | 10.0 |
| (microcrystalline cellulose) | |
| SLS | 2.25 |
| Plasdone K29-32 (PVP) | 6.53 |
| Sicovit Yellow 10 E 172 | 0.08 |
| External Phase | |
| Aerosil 200 (silicon dioxide) | 0.39 |
| Magnesium stearate | 0.78 |
| Total, layer | 100.0 |

### Example 8

A phase I, double-blind crossover study was performed with single oral doses of zaleplon 15 mg in three formulations (A, B, C) with different release characteristics; placebo; and an open comparator arm (immediate-release commercial zaleplon, 10 mg). Nineteen healthy volunteers (13 female, 6 male; ages 21-46) received treatments separated by a 4 to 7 day washout period. Blood samples were drawn predose and at 13 time points up to 12 hours postdose. Noncompartmental analysis was performed on the samples to calculate pharmacokinetics including:
peak plasma concentration (“Cmax”);
time from administration to Cmax (“Tmax”);
time from administration to drug release (“lag time”);
elimination half-life (“T½”);
and area under the plasma concentration-time curve to the time of last quantifiable concentration (“AUC”).

The results are included in Table 10:

TABLE 10

| | Formulation | | | |
|---|---|---|---|---|
| | Immediate | | | |
| | Release | A | B | C |
| Relative | | 98% | 97% | 93% |
| bioavailability | | | | |
| Lag time | 0 | 3.1 ± 0.3 | 1.9 ± 0.3 | 1.2 ± 0.5 |
| (hours ± SD) | | | | |
| Tmax | 1.5 ± 0.8 | 4.9 ± 1.0 | 4.1 ± 0.7 | 3.9 ± 0.9 |
| (hours ± SD) | | | | |
| T½ | 1.2 ± 0.2 | 1.5 ± 0.3 | 1.4 ± 0.4 | 1.8 ± 0.4 |
| (hours ± SD) | | | | |
| AUC | 56.8 ± 2.60 | 83.2 ± 53.0 | 83.1 ± 45.7 | 79.5 ± 57.0 |
| (ng · | | | | |
| h/mL ± SD) | | | | |

No differences were noted between males and females.

The A, B, and C formulations of zaleplon provided consistent active drug concentrations at different time points after administration with rapid decline after Tmax. Pharmacokinetics profiles differed between formulations and the active comparator, but were similar within treatment arms.

### Example 9

Three formulations of zaleplon were studied in healthy volunteers to determine pharmacodynamic profile (“PD”) over a 12-hour period post-dosing.

Non-elderly adults were enrolled in a cross-over, double-blind trial. Objective measures of PD were obtained by 4-lead (F4-T4, F3-T3, T4-O2, T3-O1) electroencephalography (“EEG”) and the Karolinska Drowsiness Test (“KDT”). EEG and KDT were obtained 1 hour pre-dose (baseline), and at each hour post-dose after receiving single oral dose of each release formulation (A, B, C) of zaleplon (15 mg), placebo, or marketed zaleplon (10 mg). EEG parameters were calculated on the median of the 4 leads for the standard EEG and for each 3 derivations (Fz-Cz, Cz-Pz, Pz-Oz) for the KDT during eyes-open and eyes-closed sessions. Results for EEG and KDT at each time point were expressed as change from baseline. Drug plasma levels were obtained at the same times.

18 subjects (12 females, 6 males, ages 21-46) had available data. Alpha-Slow wave Index (“ASI”), absolute power in the alpha band, and total absolute power varied significantly as a function of treatment (p<0.001, p<0.001, p=0.008, respectively). Formulations A, B, and C globally decreased these parameters 3, 4 and 5 hours after administration compared to placebo and zaleplon. KDT parameters correlated with EEG with the greatest sleepiness generally noted at the same periods of time. Results for EEG and KDT corresponded to drug plasma levels, which peaked between 3.9 and 4.9 hours post-dose for the three 15 mg formulations and 1.5 hours for zaleplon 10 mg. EEG and KDT parameters were comparable to placebo 8 hours post-dosing.

Therefore, it was found that zaleplon in a lag time release formulation provided maximum sedation 3 to 5 hours post-administration with no residual effects 8 hours post-dosing.

### Example 10

A phase I placebo-controlled, crossover double-blind study employed objective and subjective parameters to investigate the pharmacodynamic (“PD”) central nervous system (“CNS”) profile of three lag time formulations of zaleplon 15 mg. The results were analyzed to examine the correlation between these parameters in accurately defining the PD profile.

Nineteen healthy volunteers (13 females, 6 males; ages 21-46) received 5 study treatments: zaleplon 15 mg in formulations A, B, and C; placebo; and marketed immediate-release zaleplon 10 mg. Each treatment was separated by a 4 to 7 day washout period. Objective endpoints were changes from baseline in electoencephalography (“EEG”) calculated on the median of 4 leads for the standard EEG and for each 3 derivations (Fz-Cz, Cz-Pz, Pz-Oz) for the Karolinska Drowsiness Test (“KDT”) during eyes-open and eyes-closed sessions. Subjective endpoints included changes from baseline for the multiple step latency test (“MSLT”) and the Karolinska Sleepines Scale (“KSS”). Each test was given −20, −12, and −1 hour predose to establish baseline, and each hour for 12 hours postdose. PD CNS effects were analyzed through a 2-way mixed-moel ANOVA with treatment as a 5-level between groups factor, and as a 12-level within group factor.

The study showed a significant treatment effect for most PD endpoints. Between-treatment contrasts indicated that A, B, and C significantly (p<0.001, p<0.01, p<0.05, respectively) differentiated from the placebo for both objective and subjective evaluations of sleepiness. Treatment over time interactions were observed for the KSS and two EEG parameters (alpha slow wave index and alpha 2 absolute power). A, B, and C had a greater delayed and prolonged time course compared to immediate release zaleplon as demonstrated by all endpoints. A positive relationship between zaleplon plasma concentration and drug-related PD effects was noted with peak activity 4 to 5 hours postdose.

Therefore, the study showed that the PD profile of three lag time formulations of zaleplon was consistent as defined by objective and subjective evaluations.

### Example 11

In a phase I trial, the Addiction Research Center Inventory (“ARCI-49”) and the Karolinska Sleepiness Scale (“KSS”) were administered in order to measure changes in subject-perceived alertness after administration of three formulations of zaleplon. The study included a double-blind, crossover, placebo and marketed immediate-release zaleplon (10 mg) controlled study, which compared three formulations (A, B, C) of zaleplon (15 mg) in healthy volunteers. Nineteen subjects (13 female, 6 male; aged 21-46) were tested. The ARCI-49, a self-rating 49-item true-false questionnaire, measure subjective effects of drugs with diverse pharmacological actions. Sedation subscale data are presented here. The KSS, a nine-point self-rating Likert scale (1=very alert and 9=very sleepy) was also performed. Both scales were presented one hour before administration (baseline). ARCI-49 was administered 1, 3, 5, and 8 hours postdose; KSS was administered every hour for 12 hours postdose.

The results of the ARCI-49 showed that subjects felt significantly more sedated 1 hour after receiving control zaleplon compared with A (p=0.0048), B (p<0.001), or C (p=0.012). The KSS test showed that the A, B, and C formulations increased subjective sleepiness versus the placebo (p<0.001, p=0.0197, and p=0.0261, respectively versus the placebo); the time course and amplitude of the effect were different between formulations. Compared to zaleplon, all three formulations led to greater subjective feelings of sleepiness at later time points following administration.

Both subjective scales led to the same observation: a significant increase in subjective sedation and sleepiness feelings was noticed under all three formulations. Compared to immediate-release zaleplon, these increases occurred later with the new formulations of zaleplon.

### Example 12

A formulation was analyzed for solubility using various media for dissolution. The media used were:

(1) water and 0.02% SLS;

(2) acetate buffer pH=4.5, and

(3) water.

TABLE 1
Solubility test performed at 37 ± 0.5° C.

| Time | | | |
|---|---|---|---|
| (Hours) | Water | Water + 0.02% SLS | 50 mM Acetate buffer pH 4.5 |
| 1 | 0.28 | 0.28 | 0.28 |
| 2 | 0.28 | 0.28 | 0.28 |
| 4 | 0.27 | 0.28 | 0.28 |
| 24 | 0.28 | 0.28 | 0.28 |

TABLE 2
Solubility test performed at room temperature

| Solvent | Solubility (mg/ml) |
|---|---|
| water | 0.20 |
| 0.1M HCl | 0.20 |
| 0.05M acetate buffer pH 4.5 | 0.20 |
| 0.05M phosphate buffer pH 4.5 | 0.18 |
| 0.05M phosphate buffer pH 6.8 | 0.18 |
| 0.05M phosphate buffer pH 3.0 | 0.18 |
| 0.05M phosphate buffer pH 6.0 | 0.18 |
| 0.05M phosphate buffer pH 8.0 | 0.17 |
| 0.05M phosphate buffer pH 10.0 | 0.17 |
| 0.05M phosphate buffer pH 12.0 | 0.16 |

The analysis demonstrated the same or substantially the same solubility resulted regardless of the dissolution medium.

We claim:

1. A method of treating insomnia comprising administering to a subject a formulation comprising zaleplon, wherein the formulation is adapted to:
release the zaleplon after a lag time of at least about one hour after administration of the formulation, and during which substantially no drug substance is released;
provide a time of peak plasma concentration of about 3 hours to about 6 hours after administration;
provide an elimination half-life after the time of peak plasma concentration of about 0.5 hours to about 0.3 hours; and
provide an area under the curve of about 70 ng·h/mL to about 90 ng·h/mL.

2. The method of claim 1, wherein the lag time is at least about 1.5 hours.

3. The method of claim 1, wherein the time of peak plasma concentration is about 3.75 hours to about 5.25 hours after administration.

4. The method of claim 1, wherein the time of peak plasma concentration is about 4 hours to about 5 hours after administration.

5. The method of claim 1, wherein the elimination half-life is about 0.5 hours to about 2.5 hours.

6. The method of claim 1, wherein the elimination half-life is about 1 hour to about 2 hours.

7. The method of claim 1, wherein the area under the curve is about 75 ng·h/m to about 85 ng·h/mL.

8. The method of claim 1, wherein the area under the curve is about 78 ng·h/mL to about 85 ng·h/mL.

9. The method of claim 1, wherein the formulation provides maximum sedation about 3 hours to about 5 hours after administration of the formulation.

10. The method of claim 1, wherein less than about 10% of the zaleplon is released during the lag time.

11. The method of claim 1, wherein the formulation provides no residual side effects about 8 hours post-dosing.

12. The method of claim 1, wherein the formulation comprises a core and a shell.

13. The method of claim 12, wherein the core comprises zaleplon, hydroxypropylmethyl cellulose, and lactose monohydrate.

14. The method of claim 12, wherein the core comprises about 20% to about 30% zaleplon.

15. The method of claim 12, wherein the core comprises about 25% zaleplon.

16. The method of claim 12, wherein the core comprises about 25% to about 35% hydroxypropylmethyl cellulose.

17. The method of claim 12, wherein the core comprises about 31.4% hydroxypropylmethyl cellulose.

18. The method of claim 12, wherein the core comprises about 25% to about 35% lactose monohydrate.

19. The method of claim 12, wherein the core comprises about 31.4% lactose monohydrate.

20. The method of claim 12, wherein the core comprises about 1% to about 15% polyvinylpyrrolidone.

21. The method of claim 12, wherein the core comprises about 5% polyvinylpyrrolidone.

22. The method of claim 12, wherein the shell comprises about 35% to about 45% dibasic calcium phosphate.

23. The method of claim 12, wherein the shell comprises about 38.9% dibasic calcium phosphate.

24. The method of claim 12, wherein the shell comprises glyceryl behenate in an amount of about 15% to about 25%.

25. The method of claim 12, wherein the shell comprises glyceryl behenate in an amount of about 21.1%.

26. The method of claim 12, wherein the shell comprises about 1% to about 15% polyvinylpyrrolidone.

27. The method of claim 12, wherein the shell comprises about 6.53% polyvinylpyrrolidone.

28. The method of claim 12, wherein the shell comprises about 1% to about 15% microcrystalline cellulose.

29. The method of claim 12, wherein the shell comprises about 10% microcrystalline cellulose.

30. The method of claim 1, wherein the formulation comprises about 5 mg to about 50 mg zaleplon.

31. The method of claim 1, wherein the formulation comprises about 15 mg zaleplon.

32. A method of claim 1, wherein the formulation comprises a core and a shell, wherein the core comprises
about 20% to about 30% zaleplon;
about 25% to about 35% hydroxypropylmethyl cellulose;
about 25% to about 35% lactose monohydrate; and
about 1% to about 15% polyvinylpyrrolidone;
and wherein the shell comprises
comprises about 35% to about 45% dibasic calcium phosphate;
about 15% to about 25% glyceryl behenate;
about 1% to about 15% polyvinylpyrrolidone; and
about 1% to about 15% microcrystalline cellulose.
